(19) United States
(12) Patent Application Publication
(10) Pub. No.: US 2025/0085254 A1
(43) Pub. Date: Mar. 13, 2025

(54) SENSOR ASSEMBLY, SYSTEM, METHOD AND PH SENSOR

(71) Applicant: Analog Devices, Inc., Wilmington, MA (US)

(72) Inventors: Hari CHAUHAN, Windham, NH (US); Alexander C. STANGE, Somerville, MA (US); Mohamed AZIZE, Mohamed, MA (US)

(21) Appl. No.: 18/717,282

(22) PCT Filed: Dec. 6, 2022
(86) PCT No.: PCT/US2022/080984

§ 371 Date: Jun. 6, 2024

Related U.S. Application Data
(60) Provisional application No. 63/286,467, filed on Dec. 6, 2021.

(51) Int. Cl. G01N 27/414; G01N 27/12; G01N 27/327

(52) U.S. Cl. CPC G01N 27/4145; G01N 27/125; G01N 27/3276; G01N 27/4146

(57) ABSTRACT

The present disclosure provides a sensor assembly for detecting the presence of at least one amplification product of an isothermal amplification process (i.e., an isothermal nucleic acid amplification process). The sensor assembly includes a first sensor responsive to the detection of an indicator of the presence of at least one amplification product to provide a first signal, the first sensor comprising a sensing surface arranged to contact a sample in the amplification product receiving region, the sensing surface comprises a first layer comprising a one-dimensional or two-dimensional material. Alternatively or additionally, the disclosure provides a pH sensor comprising a sensing surface comprising a first layer comprising a one-dimensional or two-dimensional material.

### CROSS-REFERENCE TO RELATED APPLICATIONS

This application is a 35 U.S.C. § 371 National Stage Application of International Patent Application No. PCT/US2022/080984, filed Dec. 6, 2022, which claims the benefit of and priority to U.S. Provisional Patent Application No. 63/286,467, filed Dec. 6, 2021, the contents of which applications are hereby incorporated by reference herein in their entireties.

### FIELD OF THE DISCLOSURE

This disclosure relates to a sensor assembly for detecting the presence of at least one amplification product of an isothermal amplification process, a system, a method and a pH sensor.

### BACKGROUND

Isothermal amplification assays are used to detect particular nucleic acid sequences in a sample, for example in a sample containing a virus. These assays use isothermal (e.g. fixed temperature) amplification processes to amplify the amount of the nucleic acid sequences or a derivative thereof (both referred to herein as “amplification product(s)”) so that the presence of the amplification product can be detected by a detector or sensor, and the presence of the particular nucleic acid sequence inferred. These techniques have advantages over methods such as traditional polymerase chain reaction (PCR)-based amplification methods since they do not require thermal cycling, and instead are carried out at a constant temperature. As a result, these techniques require less complex equipment lend themselves to point-of-care and at-home testing.

However, existing isothermal assay systems and kits suffer from a number of drawbacks, in part due to the way in which the amplification product(s) are detected. In particular, these can be slow to provide a result and expensive to produce. For example, detection often relies on the use of optical dyes which can take ˜30 minutes to develop in the presence of the amplification products. These optical dyes then must be detected using an optical detector, which often use light sources and detectors requiring expensive and bulky equipment.

It would be useful to provide improved isothermal amplification assays which are quicker, cheaper and more suited to point-of-care and home testing environments.

### SUMMARY OF THE DISCLOSURE

The present disclosure provides a sensor assembly for detecting the presence of at least one amplification product of an isothermal amplification process (i.e. an isothermal nucleic acid amplification process). The sensor assembly includes a first sensor responsive to the detection of an indicator of the presence of at least one amplification product to provide a first signal, the first sensor comprising a sensing surface arranged to contact a sample in the amplification product receiving region, wherein the sensing surface comprises a first layer comprising a one-dimensional or two-dimensional material. Alternatively or additionally, the disclosure provides a pH sensor comprising a sensing surface comprising a first layer comprising a one-dimensional or two-dimensional material.

In one embodiment, a sensor assembly for detecting the presence of at least one amplification product of an isothermal amplification process comprises: an amplification product receiving region; and a first sensor responsive to the detection of an indicator of the presence of at least one amplification product to provide a first signal, the first sensor comprising a sensing surface arranged to contact a sample in the amplification product receiving region. The sensing surface comprises a first layer comprising a one-dimensional or two-dimensional material. The first sensor is either (i) a pH sensor adapted to detect a change in pH indicative of the presence of at least one amplification product; or (ii) a sensor adapted to detect the presence of at least one amplification product directly.

In one embodiment, a system for isothermal amplification of a sample comprises an isothermal amplification assembly for carrying out an isothermal amplification process on the sample to produce at least one amplification product; and a sensor assembly for detecting the presence of the at least one amplification product, the sensor assembly comprising: an amplification product receiving region; and a first sensor responsive to the detection of indicator of the presence of at least one amplification product to provide a first signal, the first sensor comprising a sensing surface arranged to contact a sample in the amplification product receiving region, wherein the sensing surface comprises a first layer comprising a one-dimensional or two-dimensional material.

In one embodiment, a method for determining the presence of an analyte in a sample, the method comprises: subjecting the sample to an isothermal amplification process, the isothermal amplification process producing at least one amplification product in the event that the analyte is present; providing the at least one amplification product to an amplification product receiving region; and detecting the presence of the at least one amplification product using a sensor assembly, the sensor assembly comprising a first sensor responsive to the detection of indicator of the presence of at least one amplification product to provide a first signal, the first sensor comprising a sensing surface arranged to contact a sample in the amplification product receiving region, wherein the sensing surface comprises a first layer comprising a one-dimensional or two-dimensional material.

In one embodiment, a pH sensor comprises a sensing surface arranged to contact a sample, the sensing surface comprising a first layer comprising a one-dimensional or two-dimensional material, wherein pH sensor is adapted to provide a signal indicative of the pH of a sample.

### BRIEF DESCRIPTION OF THE DRAWINGS

The present invention will now be described in more detail with reference to the accompanying drawings, which are not intended to be limiting:

FIG. 1A provides a schematic plan view of a sensor assembly according to an embodiment;

FIG. 1B provides a schematic cross-sectional view of the sensor assembly of FIG. 1A;

FIG. 2 provides a schematic plan view of a system according to an embodiment;

FIG. 3 provides a schematic cross-sectional view of a sensor assembly according to an embodiment;

FIG. 4 provides a schematic cross-sectional view of a sensor assembly according to an embodiment;

FIGS. 5A and 5B provide a schematic plan and schematic cross-sectional view, respectively, of a system according to an embodiment; and

FIGS. 6A and 6B provide schematic graphs of responses of a negative sample and a positive sample, respectively.

### DETAILED DESCRIPTION

Isothermal amplification assays are used to detect particular nucleic acid sequences in a sample, for example in sample containing a virus. These use isothermal amplification processes to amplify the amount of the nucleic acid sequences or a derivative thereof (both of which are referred to independently or together as “amplification product(s)” herein) so that the presence of the amplification product(s) can be detected by a detector or sensor, and the presence of the particular nucleic acid sequence inferred. These techniques have advantages over methods such as typical polymerase chain reaction (PCR) since they do not require thermal cycling, and instead are carried out at a constant temperature. As a result, these techniques lend themselves to point-of-care and at-home testing.

The present disclosure provides a sensor assembly for detecting the presence of at least one amplification product of an isothermal amplification process (i.e. an isothermal nucleic acid amplification process). The sensor assembly includes a first sensor responsive to the detection of an indicator of the presence of at least one amplification product to provide a first signal, the first sensor comprising a sensing surface arranged to contact a sample in the amplification product receiving region, the sensing surface comprises a first layer comprising a one-dimensional or two-dimensional material. Alternatively or additionally, the disclosure provides a pH sensor comprising a sensing surface comprising a first layer comprising a one-dimensional or two-dimensional material.

In one embodiment, a sensor assembly for detecting the presence of at least one amplification product of an isothermal amplification process comprises an amplification product receiving region; and a first sensor responsive to the detection of an indicator of the presence of at least one amplification product to provide a first signal, the first sensor comprising a sensing surface arranged to contact a sample in the amplification product receiving region. The sensing surface comprises a first layer comprising a one-dimensional or two-dimensional material. The first sensor is either (i) a pH sensor adapted to detect a change in pH indicative of the presence of at least one amplification product; or (ii) a sensor adapted to detect the presence of at least one amplification product directly.

The use of such a sensor assembly provides an improved means of detecting the presence of a particular nucleic acid sequence in a sample. The use of the disclosed transducers (e.g. electrochemical sensors or chemiresistors) as the first sensor enables quick yet precise detection of the amplification product(s) or an indicator of the presence of the amplification product(s), which provides a faster indication of whether the target of interest (e.g. a particular nucleic acid sequence) is present.

The presence of the first layer, which comprises (or in some embodiments is formed of) a one-dimensional (1D) or two-dimensional (2D) material, enables accurate sensing of amplification products produced in isothermal amplification techniques. Specifically, the presence of these materials is particularly advantageous as the materials themselves are responsive to electronic changes caused by the presence of molecules such as amplification products (e.g. nucleic acids or derivatives thereof). Moreover, these materials are formable into relatively thin material layers which also makes them particularly responsive to the change associated with the presence of the amplification products or an indicator thereof (e.g. a change in pH) providing a sensitive detection mechanism.

Without wishing to be bound by theory, it is also thought that the use of 1D and 2D materials as a part of a sensing surface for such a system is particularly advantageous as these have been found to operate well at the higher temperatures associated with isothermal assays. For example, it has been found that sensors using a sensing surface with a 1D or 2D material provided thereon provide sensitive and accurate detection at temperatures above room temperature (e.g., 65° C.). Conventional sensors, such as conventional pH sensors, tend not to operate accurately at these temperatures and therefore, if employed in this context, would require the solution to cool prior to testing, increasing testing time, or would sacrifice accuracy.

Additionally, the thickness and chemistry of the 1D and 2D materials means that they can act as an effective passivation layer for the sensors. This can enable further functionality, such as selectivity. The layers, including those containing graphene-like structures, can be doped or modification to provide further functionality. Hexagonal boron nitride, for example, lends itself to functionalisation and capture species can be covalently bonded to its surface. In some embodiments, the first layer isolates the transducer part of the sensor (e.g. the FET or electrode) from the liquid sample thereby reducing or eliminating errors e.g. caused by liquid disturbance. Moreover, the thickness and structure of the 2D material layers means that there is no problematic reduction in the ability of the amplification products to be detected.

Sensor assemblies and systems according to embodiments are also advantageous as the use of this type of sensor to directly detecting either the pH change (e.g. by detecting the presence of H⁺ ions) or by directly detecting the amplification product(s) themselves. Compared to prior art methods requiring development of dyes resulting from pH changes and subsequent optical detection, these provide detection in real time and thus provide a much faster result. This can dramatically reduce measurement time, and even (combined with the accuracy and sensitivity of the sensors) enables detection to take place prior to the full completion of the isothermal amplification process.

The sensor assembly provides a first sensor responsive to the detection of an indicator of the presence of at least one amplification product (e.g., the amplification product itself or a separate indicator thereof) to provide a first signal. In other words, the first sensor is response to the presence of an amplification product, for example to an indicator of the presence of an amplification product. This is when the indicator of the presence of at least one amplification product (e.g., the amplification product itself or a separate indicator thereof) is provided on the sensing surface. In other words, the first sensor is responsive to the presence of the indicator of the presence of at least one amplification product (e.g., the amplification product itself or a separate indicator thereof) to provide a first signal when said indicator is present on the sensing surface.

In the case of the first sensor being a sensor adapted to detect the presence of at least one amplification product directly, the sensor provides a rapid way of monitoring the progression of the amplification process in real time. That is, in contrast to existing methods where the amplification products then further react with other components to provide detectable outputs, this sensor can directly detect the presence of e.g. RNA/DNA to provide an indication of the presence of absence of the amplification products. This can be either through the presence of the amplification products (e.g., negatively charged RNA/DNA) near the sensing surface causing a change in the sensor (e.g. potential) or as a result of an interaction with an additional functional layer provided on the sensing surface. The latter can provide an additional level of sensitivity. These sensors have been found to be particularly well suited to detection of these products under the conditions associated with isothermal amplification (e.g. raised temperatures and in the presence of reagents).

By directly it is meant that the sensor is responsive to interacting of the amplification product with the sensing surface, rather than an indirect indicator of the presence of the amplification product. For example, this could be as a result of charge accumulation due to the presence of negatively charged amplification products on the sensing surface or may be as a result of the interaction of the amplification products with a functional component on the sensing surface (e.g., a capture species).

In the case of pH sensing, the sensor can detect the presence of H+ ions. This can be as a result of the presence of H+ ions near the sensing surface causing a change in the sensor (e.g., potential) or as a result of an interaction with an additional functional layer provided on the sensing surface. The use of such sensor in isothermal amplification assays is particularly useful as it avoids the drawbacks associated with the traditional routes for detecting the pH change, which rely on pH dyes and subsequent optical testing. In particular, the first sensor is able to accurately and directly detect the change in pH as the amplification products are created, meaning that the rate determining step is the production of the amplification products rather than the development of a dye (which is responsible for the majority of the wait time).

### One-Dimensional and Two-Dimensional Materials

By one-(1D) and two-dimensional (2D) materials, it is meant materials having nano-scale (e.g., less than or equal to 1000 nm or less than or equal to 100 nm) dimensions, with the number of dimensions above this corresponding to the name. That is, one dimensional materials can be those with only one dimension greater than nanoscale (e.g. carbon nanotubes (“CNTs”)). In the case of one-dimensional materials, these may be arranged to form the first layer, or may be provided as part of a first layer (e.g., in a matrix). Two dimensional materials have two dimensions greater than nanoscale (with one nanoscale dimension (e.g., monolayer or multilayer graphene or hexagonal-boron nitride). The first layer may comprise the two-dimensional materials or the first layer may be formed of (e.g., consist of) the two-dimensional material. A major face of the two-dimensional material may form the sensing surface.

The presence of the first layer, which comprises (or in some embodiments is formed of) a one-dimensional or two-dimensional material, enables accurate sensing of the property while also making the first sensor more robust and less prone to drift. Specifically, the presence of these materials is particularly advantageous as they protect the sensor from these liquid surface effects while also increasing sensitivity, or at the least without negatively impacting the performance of the sensor. In particular, the one- or two-dimensional material acts as a protective layer but without sacrificing the performance of the channel, due to the sensitivity of these surfaces and the structures used. The materials claimed can be provided at nano-layer thickness by using only a one or a few layers thick of the materials. This also allows them to be used more reliably for measurements which are otherwise Prone to unreliable measurements, such as pH measurements. The first layer materials provide a sensitive sensor which can detect the presence of H⁺ ions yet which does not suffer from the drawbacks of other sensors. Additionally, the thickness and chemistry of the one and two-dimensional materials means that they can act as an effective passivation layer for the sensors. The layers, including those containing graphene-like structures and CNTs, can be doped or modification to provide further functionality. Hexagonal boron nitride (hBN) and CNTs, for example, lends itself to functionalisation and capture species can be covalently bonded to its surface. The materials can also be much easier to functionalisation than existing materials. For example, in the context of a field effect transistor (FET) they provide a very thin layer which does not interfere with the channel, but which provides a surface which can be functionalised. For example, these can often be easier to functionalise than the channel materials due to the material properties and/or due to the fact that the channels are prone to damage in manufacturing processes. The presence of this first layer can therefore increase device yield and the robustness of the ultimate device.

Isothermal amplification assays also rely on heating to generate the amplification products. For example, one exemplary isothermal amplification process used in embodiments is loop assisted isothermal amplification (LAMP), which is carried out at 65° C. These materials can provide for uniform heating across the sensing surface thereby allowing the heating to occur on the surface of the sensor. Combined with the ability to directly detect the amplification products (or associated indication from the change in pH), this enables quicker results than is otherwise achievable.

In some embodiments, the one-dimensional or two-dimensional material is selected from (i.e., the first layer comprises or is formed of) graphene, hexagonal boron-nitride (hBN), carbon nano-tubes (CNTs), or a combination thereof.

In one embodiment, the one-dimensional or two-dimensional material (i.e., the first layer comprises or is formed of) is CNTs. This may include any type of CNT, such as multi-walled (including double-walled), single-walled CNTs or a combination thereof. In one embodiment, the 1D or 2D material is single-walled CNTs. These have been found to advantageously provide excellent performance (e.g., sensitivity and precision) under the conditions used in isothermal applications and for the detection of protons (e.g., in pH measurement). In some embodiments, the CNTs are semiconducting CNTs, metallic CNTs or a combination thereof, such as semiconducting, metallic single-walled CNTs or a combination thereof. The conductivity of these have been found to provide a strong response to amplification products and charged ions, such as H⁺ ions. Metallic CNTs, and more particularly metallic single-walled CNTs, have been found to be particularly advantageous in this respect. In some embodiments in which the first layer comprises or is formed of CNTs, such as single-walled CNTs, the CNTs may be functionalised. For example, they may be functionalised with polar groups such as oxygen containing groups, for example including hydroxyl (OH) or carboxylate (COOH) groups. These groups help increase the sensitivity to polar analytes, such as the amplification products (e.g. nucleic acids) and protons (for pH sensing).

Provision of CNTs as a first layer or in the first layer can be achieved using methods known in the art, such as drop casting, spray coating, dip coating or spin coating.

In one embodiment, the one-dimensional or two-dimensional material is hexagonal boron nitride (hBN). This can include hBN in its pure form, doped hBN, functionalised hBN, oxidised hBN oxide or hBN combinations thereof. hBN provides an advantageous material for use in or forming the first layer. hBN surfaces have the advantages of the first layer materials discussed above but additional are easy to functionalise. Moreover, hBN can be doped or modified to further optimise properties. The termination of the hBN surface (i.e., with N or B) can be used to further customise the properties and the ability to be functionalised. In some embodiments, hBN can act as a surface for self-assembled monolayers (SAM) on active sensing surface. hBN can also be deposited as a monolayer, or with only a few layers (e.g., 1-5, or 1-3 layers). This can provide a functional surface with little or no interference of any layers beneath, such as a channel in the context of a FET. This can accordingly act as a passivation layer. In the context of a FET, hBN, as with some other one- or two-dimensional materials has a wide-band gap (e.g., some CNTs) and can be used with lower band-gap channels to provide higher mobility and thus higher sensitivity within the channels. For example, when used in a graphene-FET (GFET), it can lead to confinement in the graphene channel.

The arrangement of the hBN layer and how the hBN is produced can influence its properties. For example, because of the heterogeneous nature of the six-membered ring, the direction of growth of the layers of hBN can influence the properties. This can result in different terminating edges (referred to as zig-zag or armchair edges). For example, in a similar manner to N-polar or Ga-Polar GaN, the different structures of hBN can be deemed to be N-polar or B-polar. N-polar and B-polar hBN have different surface properties, such as surface energies. hBN as referred to herein incorporates both N-polar and B-polar hBN. By referring to the first layer comprising N-polar hBN, this refers to the upper surface of the first layer comprising or consisting of N-polar hBN. That is, in the case of the first layer which defines the sensing surface, the outwardly facing surface of the layer relative to the remainder of the FET. In such arrangements, the face terminates in N atoms. The opposite is true for B-polar hBN (i.e. the face terminates in B atoms). In some embodiments, the first layer may comprise an N-polar outer hBN layer. That is, if the first layer comprises or consists of a single layer, the single layer is N-polar hBN, or where the first layer comprises or consists of plural layers, the layer furthest from the channel is N-polar hBN. N-polar hBN has been found to provide favourable properties due to the particular chemistry of the surface reducing the risk of undesirable interactions caused by liquid samples. This is due to electronic properties of the surface, at least in part caused by the electronegativity of N compared to B. Moreover oxidising and functionalisation are easier. Alternatively, the first layer may comprise a B-polar outer hBN layer.

In addition to graphene, other two-dimensional materials can include graphene-/graphite-like materials, such as materials having a two-dimensional planar structure sheet comprising atoms arranged in a (graphite-like or graphene-like) hexagonal formation. This includes six membered rings with sp2-hybridized carbon atoms but may include other structures, including six membered rings with atoms other than carbon. For example, this material may comprise at least one planar layer comprised of hexagonal six membered rings comprising (or consisting essentially of) carbon, boron, nitrogen and combinations thereof. This can include graphene (e.g., graphene, functionalised graphene, graphene oxide), which is a two-dimensional allotrope of carbon with a single layer of graphene includes a single planar sheet of sp2-hybridized carbon atoms. In one embodiment, the material may comprise at least one planar layer comprised of hexagonal six membered rings comprising (or consisting essentially of) at least one heteroatom and may also comprise carbon. The provision of a heteroatom (e.g., a non-carbon, non-hydrogen) in the ring structure can be advantageous as the heteroatoms (e.g., N or B) can provide a site to bond further components to the surface (e.g., during functionalisation), enabling the further customisation. For example, this can act as a surface for self-assembled monolayers (SAM) on the active sensing surface.

In some embodiments, the first layer is formed of the one-dimensional or two-dimensional material. That is, in some embodiments, the first layer consists of the one-dimensional or two-dimensional material.

The sensor assembly incorporating these transducers can be amenable to time- and cost-effective microfabrication using photolithography without the need for manual assembly of discrete components, such as light sources and LED components, thereby simplifying the fabrication process. The sensors can also be produced using low-cost monolithic integration of sensors on e.g. CMOS Si electronics wafers or plastic wafers. Moreover, conventional thin backside-illuminated (BSI) photodiode wafers typically require a carrier wafer for handling to prevent breakage. They also, often, require an expensive ceramic substrate in the assembled solution. The sensor assembly can avoid using BSI photodiode wafers to implement an optical sensor and hence avoid these issues.

The one or two-dimensional materials can include at least 1 atomic layer of said material and may be up to or equal to 15 atomic layers of said material. The first layer can accordingly be 1 to 15 atomic layers thick.

In some embodiments, the first layer may be less than 10 nm thick, such as less than 5 nm thick. In embodiments, the first layer may have a thickness of from 0.1 nm to 10 nm, such as 0.1 nm to 5 nm, such as 1 nm to 10 nm or 1 nm to 5 nm. Thickness can be determined by scanning electron microscopy (SEM), for example, and may refer to the average thickness (e.g., mean) or may refer to a maximum thickness. At these thicknesses, the first layer is particularly responsive.

In some embodiments, the first layer may be doped. For example, the one-dimensional or two-dimensional material may be doped to improve the ability of the surface to be functionalised. In one embodiment, the material may be doped with metal atoms. For example, gold-doped graphene. It has been found that gold can be used to covalently bond capture species to the sensing surface (e.g. to the graphene). This could be achieved using thiol-terminated capture specifies, such as DNA- or RNA-based aptamers or probes.

In some embodiments, the first layer may comprise plural layers, such as a plurality of layers of 1D and/or 2D materials. For example, in one embodiment, the first layer may comprise a graphene layer and a hBN layer provided on top of the graphene layer. This may lead to improved sensor arrangements, whereby the most sensitive materials can be combined with those which also provide high performance, but which enable other modifications. For example, in the case of a graphene layer and a hBN layer provided on top of the graphene layer, the hBN can allow for further functionality to be added to the sensing surface by virtue of the heteroatoms present in its structure.

### Transducers

The sensing assembly may be for detecting or configured to detect the presence of at least one amplification product of an isothermal amplification process a property of (e.g., a parameter of or analyte in) a sample. The sample may be a fluid sample (e.g., a liquid sample).

For example, the sensors of the sensing assembly may be used in embodiments for determining (or configured to determine) the concentration of at least one amplification product of an isothermal amplification process and/or pH (determination of the −log 10 molar concentration of H⁺ ions). The term “concentration” as used herein may, in certain embodiments, refer to the activity of the analyte in question (e.g., amplification product or protons). In some embodiments, a first sensor may be configured to detect the concentration of at least one amplification product of an isothermal amplification process and a second sensor may be configured to detect the pH.

Detection of the amplification product or protons (i.e., the analytes in question) may be achieved by directly sensing the specific analyte. By directly it is meant that the sensing assembly is responsive to the interaction between the analyte with the sensing surface, rather than an indirect indicator of the presence of the analyte. For example, this could be as a result of charge accumulation due to the presence of the analyte (if charged) or may be as a result of the interaction of the analyte with a functional component on the sensing surface (e.g., a capture species and/or functional groups).

In the case of pH sensing, the sensing assembly can (e.g., can be configured to) detect the presence of protons (i.e., H⁺ ions). This can be as a result of the presence of H⁺ ions near the sensing surface causing a change in the channel or as a result of an interaction with an additional functional layer provided on the sensing surface. pH sensing as set out herein can, in embodiments, be relative pH sensing. That is, the system may provide an indication of the change of pH from a baseline rather than an absolute value. This reduces the complexity of the sensor while still providing a means for determining the outcome of the isothermal assay.

In embodiments, the sensing assembly is configured for analysing a body fluid. In embodiments, this can be blood, urine, mucus, or saliva.

The use of transducers of the disclosed type can be particularly advantageous. In embodiments, the sensor assembly comprises electrode-based sensors and FET-based sensors (including e.g. MOSFET-based sensors). In one embodiment, the electrode-based sensor is a chemiresistors.

In some embodiments, the first sensor comprises is an electrode-based sensor comprising at least one electrode adapted to provide the first signal. The first layer may be provided on a surface of a first working electrode to provide the sensing surface. For example, the first sensor may comprise an electrode on top of which is provided the first layer. Further functionalisation may be provided on the first layer and/or the surface of the electrode. Changes in or interactions with the functional layer formed on the surface can be detected through changes in potential. For example, the molecules adhered to the surface may change position relative to the sensing surface and accordingly change the sensing potential. In other cases, the binding of analyte to the surface may result in a change in potential. Examples of the types of molecules which are commonly employed for this mode of sensing mechanism include aptamers and nucleic acids. These molecules may also be tagged with a redox label such as methylene blue. These may be sensed by any suitable method of transduction. For example, this may comprise measuring the potential (e.g., voltage), current, permittivity, charge and/or frequency. In some embodiments, changes in or interactions with the first layer on the sensing surface can be detected through changes in potential. In other embodiments, changes may be determined by monitoring changes in current passing through each sensing site (at a constant potential).

In some embodiments, the first layer may extend between two electrodes. For example, the electrodes may be spaced apart) with the first layer extending therebetween. The electrodes may measure a change in resistance of the first layer to provide a chemiresistor. In some embodiments, there may be two electrodes (two-terminal) and in other embodiments there may be more than two electrodes. These have been found to be a particularly advantageous as these can readily detect the changes in the first layer as a result of interaction of the material of the first layer with the amplification products/protons, as well as being relatively easy to manufacture. For example, as compared to a field effect transistor (FET). In embodiments, the first layer may have a thickness of from 0.1 nm to 10 nm, such as 0.1 nm to 5 nm, such as 1 nm to 10 nm or 1 nm to 5 nm. In embodiments providing a chemiresistor, it has been found that advantageous first layers provide a resistance of from 1 ohm to 1 Kohm between the electrodes.

Where electrode(s) are used, these may comprise or be formed from metals, metal oxides, metal nitrides, carbon-based materials, a conductive polymer, doped silicon or polysilicon or combinations thereof. In an embodiment, the electrodes may be formed from or comprise gold, silver, copper, platinum, nickel, titanium, titanium nitride, ruthenium, ruthenium oxide or combinations thereof.

Alternatively, the first sensor comprises a field effect transistor (FET) adapted to provide the first signal, and the first layer is provided on a surface of the FET. A FET may use a gate oxide layer as a part of the sensing surface (e.g., a base), with the first layer provided thereon. The first layer may therefore act as a capping or passivation layer on the FET. It has been found that this can greatly improve the functionality of the FET, as the first layer may improve the mobility of the channel of the FET, as the one- or two-dimensional materials constrain flow of charge carriers through the channel. This in turn provides high sensitivity, reducing the amount of amplification product needed before it can be detected.

For example, in some embodiments, the first sensor can be a FET comprising the first layer providing a sensing surface; a channel provided below the first layer; and a drain and a source in electrical communication with the channel. The sensing assembly may further comprise a gate provided below the first layer and the first layer comprises a one-dimensional or two-dimensional material. The FET may therefore provide a three-terminal structure. The 1D or 2D material can be any of those listed herein. In some embodiments, the first layer comprises or is formed of hBN (such as N-polar hBN and/or hBN on a layer of graphene) or CNTs. In some embodiments, the CNTs of the first layer of the FET are semiconducting CNTs, metallic CNTs or a combination thereof, such as semiconducting, metallic single-walled CNTs or a combination thereof. These materials have been found to provide particularly effective sensors. Without wishing to be bound by theory, these materials have been found to provide the advantages discussed herein, with the additional benefit of providing a wide band gap (compared to the channels) thereby increasing confinement of charge within the channel. In some embodiments, the FET is a graphene FET (i.e. with a graphene channel), where this confinement effect is pronounced.

Additionally, the thickness and chemistry of the materials means that they can act as an effective passivation layer for the FET. The layers, including those containing graphene-like structures, can be doped or modification to provide further functionality. In some embodiments, the first layer isolates the gate dielectric from the liquid sample thereby reducing or eliminating errors e.g. caused by liquid disturbance. Moreover, the thickness and structure of the 2D material layers means that there is no problematic reduction in the ability of the amplification products to be detected by the FET (transport properties remain unaffected).

In some embodiments of the FET, the first sensor may further comprise a second layer beneath the channel comprising or being formed of a one dimensional or two-dimensional material. This can be used to further constrain charge carrier movement to the channel.

In one embodiment, the first sensor is a FET, and the first layer comprises or is formed of hexagonal-boron nitride. This has been found to be an advantageous combination, since the hBN layer (e.g. N-polar or B-polar hBN) has been found to act as an excellent wide-band gap layer. In some embodiments, the first sensor may further comprise a second layer beneath the channel comprising or being formed of hexagonal-boron nitride. This further constrains the charge carriers.

In one embodiment, the FET is a graphene-FET (i.e., the channel is formed of graphene), and the first layer is a hBN layer formed on the FET. The hBN has been found to boost the mobility (up to 10×) of the graphene channel, which results in improved sensitivity and a higher overall performance. The layer also improves the customisability, since the hBN is more amenable to functionalisation as compared to graphene and can be covalently bonded to capture species. This improves the manufacturability of the device.

The specific FET structures discussed in the embodiments above are also all amenable the sensor is amenable to time- and cost-effective microfabrication using photolithography and without the need for manual assembly of discrete components (e.g., electrodes), thereby simplifying the fabrication process.

### Sensing Surface

The sensing surface may provide a receiving region for a fluid (e.g., a liquid). This may be a receiving region for the amplification products (e.g., after the isothermal amplification is carried out) or may be a receiving region for the sample and reagents, with the isothermal amplification assay carried out thereon. In some embodiments, a reaction chamber may be provided on (e.g., directly on) the sensing surface and may be fluidly connected to the sensing surface (e.g. open to the sensing surface) so that the first sensor can detect the presence of any amplification products or a change in pH in situ as the amplification progresses. Where plural sensors are used, the reaction chamber may be provided on (e.g., directly on) each of these and may be fluidly connected to the sensing surface of each of these.

In one embodiment, the sensing surface is functionalised. This can be with an interaction or capture species. Such functionalization can be achieved in any suitable manner, such as by covalently or non-covalently immobilizing the interaction or capture species to the surface. Interaction or capture species are configured to selectively interact with an analyte. In this instance, this may be with the DNA or RNA (i.e., DNA- or RNA-based aptamers or probes). This can be achieved, for example, using an aptamer. In some non-limiting examples, the aptamer is functionalized with an electro-active moiety, for example a redox-active moiety, and is configured such that a conformational change of the aptamer upon selectively interacting with, for example binding, the analyte causes a change in the proximity of the electro-active moiety with respect to the surface of the respective working electrode. Particularly in examples in which the working electrode is configured for determining a change in current associated with the selective interaction with the analyte, such a change in proximity of the electro-active moiety with respect to the surface of the respective test electrode can cause, or at least contribute to, the determined current change. Thus, the aptamer being functionalized with such an electro-active moiety can assist with amperometric sensing of the analyte.

### Second Sensor

In one embodiment, the sensor assembly of any preceding aspect, further comprising a second sensor responsive to the detection of an indicator of the presence of at least one amplification product to provide a second signal; and wherein the first sensor is a pH sensor adapted to detect a change in pH indicative of the presence of at least one amplification product and the second sensor is a sensor adapted to detect the presence of at least one amplification product directly. In other words, the sensor assembly can include both sensors (i) and (ii). One or both of the sensors may comprise a sensing surface configured as disclosed herein (i.e., comprising a one-dimensional or two-dimensional material, as configured according to any of the embodiments set out herein).

Such a system is particularly advantageous as it provides a dual sensing ability, providing confirmation of the detection of the amplification products. This improves the accuracy of the assay and reduces the likelihood of false positives.

Moreover, the use of the specific disclosed sensors is particularly advantageous as both provide rapid and real-time results which can provide the dual confirmation significantly faster (e.g. <1 minute) than existing single-detector systems. This also allows for less selective measurement techniques to be used, as the use of a dual sensor assembly will provide confirmation via another detection method and thus provide some degree of selectivity.

In such an embodiment, the second sensor can comprise a sensing surface arranged to contact a sample in the amplification product receiving region (or a separate amplification product receiving region). The second sensor may have any of the features disclosed in respect of the first sensor. The sensing surface comprises a first layer comprising a one-dimensional or two-dimensional material.

### pH Sensing in the Sensor Assembly

In one embodiment, the first sensor is a pH sensor.

In one embodiment, the first sensor is a pH sensor and the first layer comprises (or is formed of) a carbon-based 1D or 2D material, for example in one embodiment selected from CNTs and/or graphene. The graphene can be a monolayer of graphene. The carbon-based 1D or 2D materials can be functionalised with functional groups to detect a change in pH. For example, this may be functionalised using carboxylic groups or —OH. In some embodiments, the graphene may be provided on a SiO₂ substrate. For example, the first sensor may comprise first and second electrodes between which the first layer extends, where the first layer is provided on a support or substrate for strength and durability.

In one embodiment, the first sensor is a pH sensor and the first layer comprises hBN. The hBN can be a monolayer of hBN. This can be functionalised with functional groups to detect a change in pH. For example, this may be functionalised by oxidisation, as discussed above. The disclosed sensing assemblies are particularly advantageous for pH sensing in any context due to the relative independence of the measurement on temperature. Without wishing to be bound by theory, it appears that the sensitivity provided by the presence of the first layer of the sensing surface provides a useful and reliable measurement tool for determining pH and one which is operational at all temperatures. In contrast, proper operation of conventional pH sensors can be temperature dependent.

In some embodiments, the sensor assembly may further comprise an ion selective membrane (ISE) provided on the sensing surface(s). This is located between the first layer and any sample and can be above the first layer. Such a membrane can act as a filter to prevent unwanted species from contacting the sensing surface. An ISE is not necessary in these sensors for detection and can slow the response (due to the requirement for ions to propagate through the ISE); however, it can improve accuracy of sensing and reduce interference, particularly in samples which may include high levels of non-analyte species.

### Other Sensing

In some embodiments, the sensor assembly may further comprise an optical sensor adapted to detect a change in optical properties corresponding to the presence of at least one amplification product.

### System

In one embodiment, there is provided a system for isothermal amplification of a sample, the system comprising: an isothermal amplification assembly for carrying out an isothermal amplification process on the sample to produce at least one amplification product; and a sensor assembly for detecting the presence of the at least one amplification product. The sensor assembly comprises an amplification product receiving region; and a first sensor responsive to the detection of indicator of the presence of at least one amplification product to provide a first signal, the first sensor comprising a sensing surface arranged to contact a sample in the amplification product receiving region. The sensing surface comprises a first layer comprising a one-dimensional or two-dimensional material.

The sensor assembly of this embodiment may be a sensor assembly according to any of the embodiments set out herein, and for example may comprise any of the features disclosed in respect of the first sensor (e.g. of the sensor assembly) set out in this disclosure. For example, the first sensor may be (i) a pH sensor adapted to detect a change in pH indicative of the presence of at least one amplification product; or (ii) a sensor adapted to detect the presence of at least one amplification product directly. The one-dimensional or two-dimensional material can be selected from graphene, hexagonal boron-nitride, carbon nano-tubes, or a combination thereof.

The system may further comprise a second sensor responsive to the detection of an indicator of the presence of at least one amplification product to provide a second signal; and wherein the first sensor is a pH sensor adapted to detect a change in pH indicative of the presence of at least one amplification product and the second sensor is a sensor adapted to detect the presence of at least one amplification product directly.

In one embodiment, the isothermal amplification assembly is a loop assisted isothermal amplification (LAMP) assembly. Thus the system may be a system for LAMP and the sensor assembly may be for detecting the amplification products of LAMP. LAMP is a particular type of isothermal amplification technique which can be advantageously combined with the systems and sensors set out herein. For example, LAMP is carried out at 65° C., at temperature at which the disclosed sensors have been found to be able to operate at accurately and with good sensitivity. This avoids having to wait until the reaction products have cooled to determine the presence of any amplification products.

In some embodiments, the system may further comprise a heat source, such as a heating unit, the sample and amplification reagents in the sample receiving region to a predefined temperature so as to form at least one amplification product. This can therefore be provided adjacent or abutting the first sensor (and any further sensors, where present). Provision of a heating unit in this manner can be particularly advantageous as this can allow for the isothermal amplification to be carried out in situ on the first sensor (or any other sensor where present), thereby enabling the immediate detection of any amplification products or change in pH. This is important when used with the disclosed sensors, since these are sensitive enough to detect these changes far earlier than the traditional dye-based devices. Exemplary heating units include electrical heating units.

In one embodiment, the heating source (e.g., heating unit) is provided beneath the first layer with the sensing surface on the opposite side of the first layer. This configuration has been found to be particularly effective as the heating unit can heat the entire first surface without impeding or complicating the upper sensing surface and can readily be incorporated into a substrate using common manufacturing techniques. This can be advantageously combined with arrangements in which the first layer is provided between plural electrodes. More generally, this has been found to be particularly advantageous with the first layer disclosed herein (i.e. comprising 1D/2D materials) since these have high thermal conductivity. This enables an efficient transfer of heat across the entire sensing surface (or surfaces, where plural sensors are used) and thereby heats the sample and sensor(s) uniformly.

In some embodiments, the system may further comprise a reaction chamber provided on (e.g., directly on) the sensing surface and may be fluidly connected to the sensing surface (e.g. open to the sensing surface) so that the first sensor can detect the presence of any amplification products or a change in pH in situ as the amplification progresses. Where plural sensors are used, the reaction chamber may be provided on (e.g., directly on) each of these and may be fluidly connected to the sensing surface of each of these. This can be used in conjunction with the heating source (e.g. heating unit) noted above to provide an in-situ reaction vessel which can be monitored by the sensors disclosed herein. The reaction chamber can be formed from any suitable material, such as a polymer. In one embodiment, the reaction chamber comprises polyamide, PDMS, or an ostemer (off stoichiometry thiol-ene) polymer.

### Method

In one embodiment, there is provided a method for determining the presence of an analyte in a sample, the method comprising:
a. subjecting the sample to an isothermal amplification process, the isothermal amplification process producing at least one amplification product in the event that the analyte is present;
b. providing the at least one amplification product to an amplification product receiving region; and
c. detecting the presence of the at least one amplification product using a sensor assembly, the sensor assembly comprising a first sensor responsive to the detection of indicator of the presence of at least one amplification product to provide a first signal, the first sensor comprising a sensing surface arranged to contact a sample in the amplification product receiving region, wherein the sensing surface comprises a first layer comprising a one-dimensional or two-dimensional material.

The sensor assembly of this embodiment may comprise any of the features disclosed in respect of the first sensor set out in this disclosure. In some embodiments, the method may comprise and therefore use the sensor assembly and/or system according to any of the embodiments set out herein.

In one embodiment, the method further comprises determining the presence of the at least one amplification product using a second sensor (e.g., confirming the presence). The second sensor may have the structure of any sensor set out herein (first or second).

### pH Sensor

In another embodiment, a pH sensor comprises a sensing surface arranged to contact a sample, the sensing surface comprising a first layer comprising a one-dimensional or two-dimensional material. The pH sensor is adapted to provide a signal indicative of the pH of a sample. Sensor of the sort disclosed herein are particularly advantageous for pH sensing in any context due to the relative independence of the measurement on temperature. Without wishing to be bound by theory, it appears that the sensitivity provided by the presence of the first layer of the sensing surface provides a useful and reliable measurement tool for determining pH. In contrast, proper operation of conventional pH sensors can be temperature dependent.

The pH sensor may be configured or comprise the features as set out in respect of any of the sensors disclosed herein. For example, the one-dimensional or two-dimensional material may be selected from graphene, hexagonal boron-nitride, carbon nano-tubes, or a combination thereof. In one embodiment, the first layer is formed of the one-dimensional or two-dimensional material. In one embodiment, the first layer comprises (or is formed of) a carbon-based 1D or 2D material, for example in one embodiment selected from CNTs and/or graphene. The graphene can be a monolayer of graphene. The carbon-based 1D or 2D materials can be functionalised with functional groups to detect a change in pH. For example, this may be functionalised using carboxylic groups or —OH. In one embodiment, the first layer comprises hBN. The hBN can be a monolayer of hBN. This can be functionalised with functional groups to detect a change in pH. For example, this may be functionalised by oxidisation, as discussed above.

In one embodiment, the first sensor is an electrode-based sensor comprising at least one electrode adapted to provide the first signal. In another embodiment, the first sensor comprises a field effect transistor adapted to provide the first signal, and wherein the first layer is provided on a surface of the field effect transistor.

In some embodiments, the sensor assemblies set out herein can be chip-based sensor assemblies (e.g. integrated sensor assemblies). The first, and where present second or third sensors, may be provided as part of a single chip or on a single substrate. This can facilitate manufacture and ensure that the system is more compact than existing assays.

In some embodiments, the pH sensor may further comprise an ion selective membrane (ISE) provided on the sensing surface(s). This is located between the first layer and any sample and can be above the first layer. Such a membrane can act as a filter to prevent unwanted species from contacting the sensing surface.

### Specific Embodiments

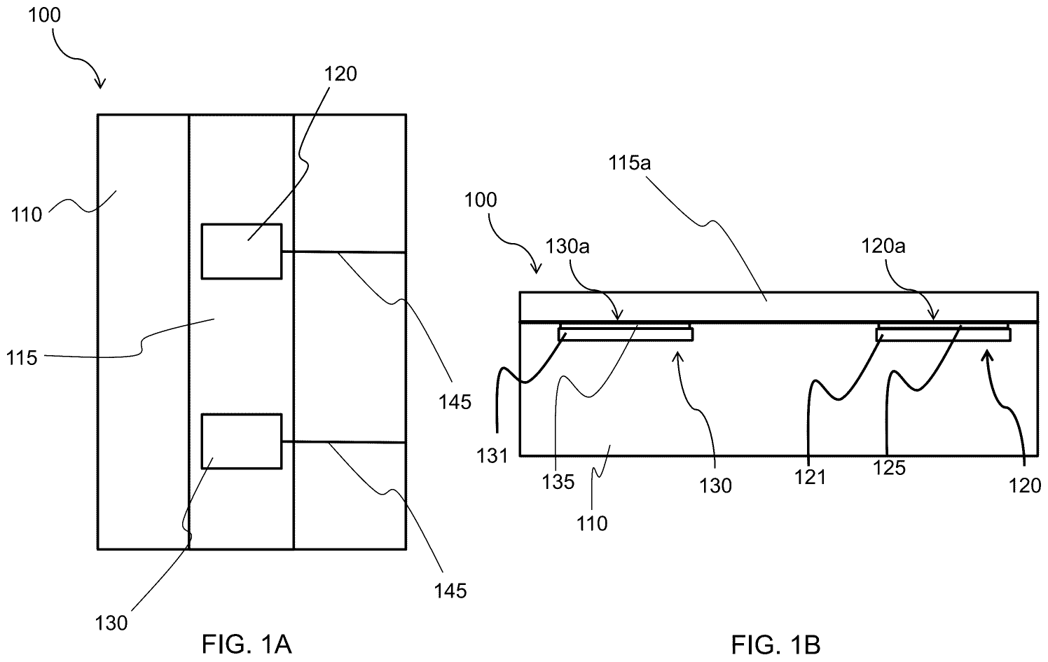

FIG. 1A depicts a schematic plan view of a sensor assembly 100 for detecting the presence of at least one amplification product of an isothermal amplification process. FIG. 1B depicts a cross-sectional view through the sensor assembly 100.

Sensor assembly 100 comprises a substrate 110, a fluid channel 115, a first sensor 120 adapted to adapted to detect the presence of at least one amplification product directly and a second sensor 130 adapted to detect a change in pH indicative of the presence of at least one amplification product. This is accordingly a dual sensor embodiment. The first and second sensors are connectable to an external signal processing system (not shown) via conductive metal tracks 145.

The sensor assembly 100 is provided with the amplification product receiving region in the form of a fluid channel 115 formed in the substrate 110 and the first and second sensors 120, 130 formed in the base of the fluid channel 115. In this way, the reaction products of the isothermal amplification can be flowed from an associated isothermal amplification assembly (not shown in FIGS. 1A and 1B), into fluid channel 115 and onto the first and second sensors 120, 130.

As noted above, the first sensor 120 in this embodiment is a sensor adapted to detect the presence of at least one amplification product directly. As depicted in FIG. 1B, the first sensor 120 is formed in the substrate 110 and provides a sensing surface 120a facing the fluid channel 115 so that fluid within the fluid channel 115 is provided on the sensing surface 120a. In this embodiment, the first sensor 120 comprises an electrode 121 on which is provided a first layer 125. These together define the sensing surface 120a. The electrode 121 of the first sensor is formed of a metal. The first layer 125 is a multilayer two-dimensional material provided on the metal. In some embodiments, this can be a layer of graphene or hexagonal-boron nitride. In this embodiment, sensing of the amplification products is achieved by sensing the negatively charged amplification products on the sensing surface 120a, the concentration of which will change as the amplification progresses (if the target is present). The change in concentration will creates a measurable change when the electrode 121 is addressed (e.g., by the external signal processing system).

The second sensor 130 in this embodiment is a pH sensor adapted to detect a change in pH indicative of the presence of at least one amplification product. As depicted in FIG. 1B, the second sensor 130 is formed in the substrate 110 and provides a sensing surface 130a facing the fluid channel 115 so that fluid within the fluid channel 115 is provided on the sensing surface 130a. In this embodiment, the second sensor 130 comprises an electrode 131 on which is provided a layer 135. These together define the sensing surface 130a. The electrode 131 of the second sensor is formed of a metal. The layer 135 is a multilayer two-dimensional material provided on the metal. In some embodiments, this can be a layer of graphene or hexagonal-boron nitride. pH sensing is achieved by sensing the presence of H+ ions in the fluid channel 115, the concentration of which will change as the amplification progresses (if the target is present). The change in concentration will create a measurable change when the electrode 131 is addressed (e.g., by the external signal processing system).

Although not shown, counter and reference electrodes are provided in this embodiment.

Such a sensor assembly 100 provides a compact, accurate and rapid means of sensing the presence of amplification products from an isothermal amplification process. Both the first and second sensors 120, 130 can provide real-time and quantitative results so that a result can be provided quickly. These can be formed using typical processing techniques, providing miniaturised sensor assemblies which are particularly well suited to home testing and point-of-care testing.

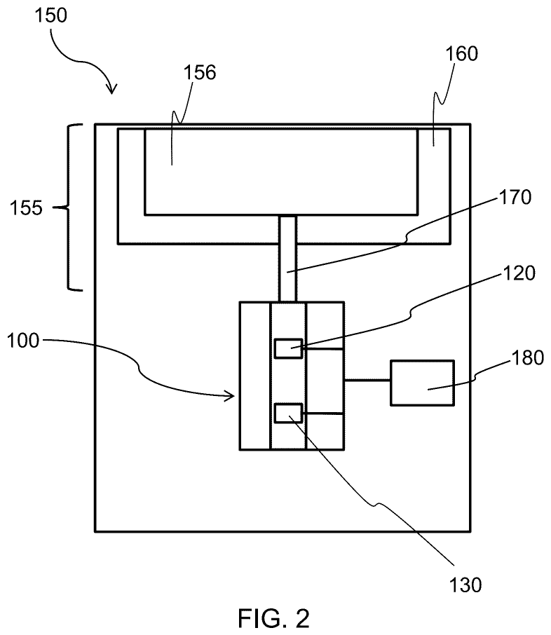

FIG. 2 provides a schematic plan view of a system 150 for isothermal amplification of a sample. The system 150 comprises an isothermal amplification assembly 155 for carrying out an isothermal amplification process on the sample to produce at least one amplification product; and a sensor assembly for detecting the presence of the at least one amplification product. In this embodiment, the sensor assembly is the sensor assembly 100 depicted in FIGS. 1A and 1B.

The isothermal amplification assembly 155 comprises a reservoir 156 which is provided with the reagents for amplifying the target nucleic acid and the sample to be analysed. The reservoir 156 is surrounded by a heating means 160 for heating the contents of the reservoir 156 to the required temperature to perform the amplification. The reservoir 156 is connected to the sensor assembly 100 via a microfluidic channel 170, which in turn connects to the fluid channel 115 of sensor assembly 100.

The first and second signals produced by the first and second sensors 120, 130 of the sensor assembly are output to a signal processing unit 180 via conductive tracks 145 so that the presence of the amplification products can be detected.

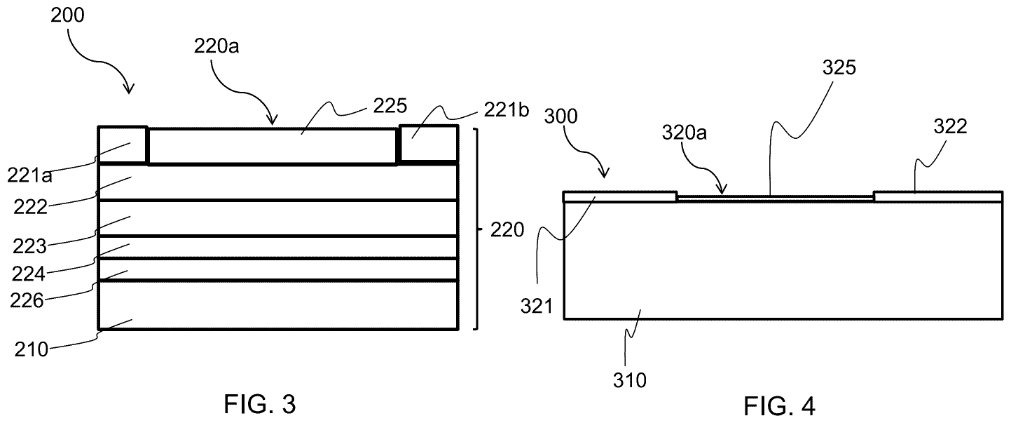

FIG. 3 provides a schematic cross-sectional view of a sensor assembly 200 for detecting the presence of at least one amplification product of an isothermal amplification process. Sensor assembly 200 comprises field effect transistor (FET) 220, which in this embodiment is a graphene-FET. As such, the channel 222 of the FET is a graphene channel. Source 221b and drain 221a electrodes a provided on the channel 222. The FET also comprises a gated-substrate 210, on which is provided (in sequential order) a gate metal 226 and a gate oxide 224.

In addition, in this embodiment, there first and second layers 225, 223 binding the channel on either side of the channel. Both of the first and second layers 225, 223 are one-dimensional or two-dimensional materials, and in this embodiment are both hexagonal-boron nitride.

The sensor assembly 200 also comprises an amplification product receiving region in the form of the upper surface which also defines the sensing surface 220a.

This arrangement has been found to be an advantageous combination, since the hBN layer (e.g., N-polar or B-polar hBN) has been found to act as an excellent wide-band gap layer. The sensor incorporating the second layer beneath the channel comprising or being formed of hexagonal-boron nitride further constrains the charge carriers and increases this effect. Overall, this has been found to boost the mobility (up to 10×) of the graphene channel, which results in improved sensitivity and a higher overall performance.

The layer also improves the customisability, since the hBN is more amenable to functionalisation as compared to graphene and can be covalently bonded to capture species. This improves the manufacturability of the device. Although not depicted in this embodiment, capture species (e.g., cDNA, Aptamers, Abs) can be applied to the surface. This may use a linker molecule such as Pbase, SH, PEG, or Biotin. In some embodiments, the hBN layer(s) can be n×hBNₓO₁₋ₓ:Si,Al,Au. When n=1×hBN, the layer can act as a passivation layer, for example.

As with the preceding embodiments, the sensor assembly 200 can be used a sensor assembly in the detection of amplification products, with the FET acting as a sensor for e.g. DNA or RNA.

FIG. 4 provides a schematic cross-sectional view of a sensor assembly 300 for detecting the presence of at least one amplification product of an isothermal amplification process.

The sensor assembly 300 comprises a substrate 310 on which is provided: a first electrode 321 on one side of an upper surface of the substrate 310, a second electrode 322 on the opposite side of the upper surface of the substrate 310 and a first layer 325. The first layer 325 extends between the first and second electrodes 321, 322 and defines the sensing surface 320a. The first electrode 321 and second electrode 322 in this embodiment are provided as metallic layers deposited on the substrate 310 and connected to associated circuitry by tracks (not shown). In this embodiment, progression of the amplification process results in changes to the resistance provided by the first layer 325. The first layer 325 comprises a one-dimensional or two-dimensional material.

In one particular embodiment, the first layer 325 comprises a graphene monolayer which is sensitive to changes in pH due to functionalisation of the graphene monolayer incorporating pH sensitive moieties on the surface. This can, in some embodiments, be a pH sensor (such as a standalone pH sensor or a pH sensor used in the detection of the amplification products discussed herein).

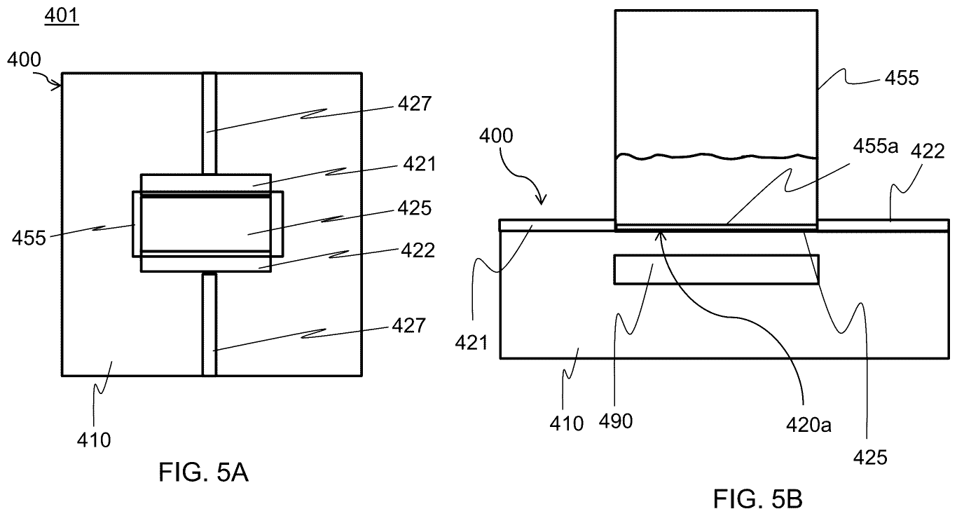

FIGS. 5A and 5B provides a schematic cross-sectional view of a system 401 comprising a sensor assembly 400 for detecting the presence of at least one amplification product of an isothermal amplification process.

In a similar manner to the structure of FIG. 4, the sensor assembly 400 comprises a substrate 410 on which is provided a first electrode 421 on one side of an upper surface of the substrate 410, a second electrode 422 on the opposite side of the upper surface of the substrate 410 and a first layer 425. The first layer 425 extends between the first and second electrodes 421, 422 and defines the sensing surface 420a. The first electrode 421 and second electrode 422 in this embodiment are provided as metallic layers deposited on the substrate 410 and connected to associated circuitry by tracks 427. In this embodiment, progression of the amplification process results in changes to the resistance provided by the first layer 425. The first layer 425 is formed of a one-dimensional or two-dimensional material.

The system 401 in this embodiment enables in situ isothermal amplification of a target nucleic acid by being configured such that the reaction to take place on the sensor assembly itself. Specifically, the system 401 further comprises a reaction chamber 455 provided on the sensing surface 420a which reaction chamber 455 receives the reagents (e.g. primer) and sample. The reaction chamber 455 is formed of a housing defining a chamber within which the fluid used in the reaction (and resulting products) can be received therein and has an open base 455a such that the fluid is in contact with the sensing surface 420a and is retained thereon. This allows the first sensor (and specifically the first layer 420) to respond to changes in the fluid, for example due to the generation of amplification products.

The system 401 in this embodiment also comprises a heating unit 490 configured to heat the fluid in the reaction chamber 455 so that the amplification process can proceed under typical conditions. In this embodiment, which can be provided underneath the first layer 425 (in the relative configuration shown in FIG. 5B) on the opposite side of the upper surface of the substrate 410. This is advantageous as it uniformly heats the reaction chamber 455 and is straightforward to manufacture, but it will be appreciated that other configurations of the heating unit 490 are possible. The 1D/2D material used in the first layer 425 is configured to thermally conduct heat across the first layer 425 so that the heating unit 490 can heat the reaction chamber 455.

Although only one sensor is depicted in FIGS. 5A and 5B, it will be appreciated that plural sensors may be provided, each having a similar or identical configuration. The plural sensors may be used for determining whether amplification products are formed for different isothermal amplification processes (e.g., performed looking for different nucleic acids), enabling the provisions of a multi-target sensor system. Alternatively or additionally, there may be plural sensors provided to increase reliability and accuracy, for example to provide controls (such as a positive control and a negative control).

In one non-limiting example of the embodiment of FIGS. 5A and 5B, an advantageous configuration of the system 400 has a first layer formed of metallic single-walled CNTs. This provides an effective first layer 425 for the reasons set out above, and moreover as it provides high thermal conductivity between the heating unit 490 and the reaction chamber 455.

### Example

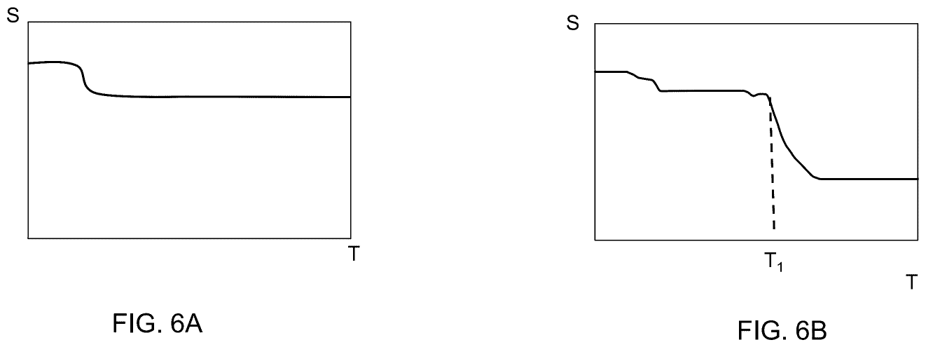

FIGS. 6A and 6B provides a graph of sensor response using a system in line with system 401 of FIGS. 5A and 5B and with a first layer consisting of metallic single-walled CNTs provided with a thickness of ˜3 nm and. FIG. 6A provides sensor response vs time for a negative control and FIG. 6B shows sensor response vs time for a positive sample. As can be seen, there is a large response at T₁ which corresponds to the production of the amplification products and a change in pH resulting from this. Before this, the response is a baseline response. T₁ is significantly lower (i.e., sooner) for this experiment performed in situ than for those relying on optical sensing and pH dyes, for example, and can be 10 minutes (i.e., corresponding to the time taken for the amplification products to form) or less, in some cases less than 5 minutes and in some cases even 60 seconds. That is, the sensor assemblies and systems of the present embodiments are limited primarily by the speed of the amplification reaction, and not subsequent detection. Moreover, the first layers provide a clear and detectable response which can be distinguished from negative samples with increased accuracy.

It should be understood that the detailed description and specific examples, while indicating exemplary embodiments of the apparatus, systems and methods, are intended for purposes of illustration only and are not intended to limit the scope. These and other features, aspects, and advantages of the apparatus, systems and methods of the present invention can be better understood from the description, appended claims or aspects, and accompanying drawings. It should be understood that the Figures are merely schematic and are not drawn to scale. It should also be understood that the same reference numerals are used throughout the figures to indicate the same or similar parts.

Other variations to the disclosed embodiments can be understood and effected by those skilled in the art in practicing the disclosure, from a study of the drawings, the disclosure, and the appended aspects or claims. In the aspects or claims, the word “comprising” does not exclude other elements or steps, and the indefinite article “a” or “an” does not exclude a plurality. The mere fact that certain measures are recited in mutually different dependent aspects or claims does not indicate that a combination of these measures cannot be used to advantage. Any reference signs in the claims should not be construed as limiting the scope.

### Select Examples

Example 1 provides a sensor assembly for detecting the presence of at least one amplification product of an isothermal amplification process, the sensor assembly comprising: an amplification product receiving region; and a first sensor responsive to the detection of an indicator of the presence of at least one amplification product to provide a first signal, the first sensor comprising a sensing surface arranged to contact a sample in the amplification product receiving region, wherein the sensing surface comprises a first layer comprising a one-dimensional or two-dimensional material; and wherein the first sensor is either (i) a pH sensor adapted to detect a change in pH indicative of the presence of at least one amplification product; or (ii) a sensor adapted to detect the presence of at least one amplification product directly.

Example 2 provides the sensor assembly of example 1, wherein the one-dimensional or two-dimensional material is selected from graphene, hexagonal boron-nitride, carbon nano-tubes, or a combination thereof.

Example 3 provides the sensor assembly of example 2, wherein the one-dimensional or two-dimensional material is single-walled carbon nanotubes.

Example 4 provides the sensor assembly of any of examples 1 to 3, wherein the first layer is formed of the one-dimensional or two-dimensional material.

Example 5 provides the sensor assembly of any preceding example, wherein the first sensor is an electrode-based sensor comprising at least one electrode adapted to provide the first signal.

Example 6 provides the sensor assembly of any of examples 1 to 4, wherein the first sensor comprises a field effect transistor (FET) adapted to provide the first signal, and wherein the first layer is provided on a surface of the FET.

Example 7 provides the sensor assembly of example 6, wherein the first sensor further comprises a second layer beneath a channel of the field effect transistor, the second layer comprising or being formed of a one dimensional or two-dimensional material.

Example 8 provides the sensor assembly of one of example 6 or example 7, wherein the first layer comprises or is formed of hexagonal-boron nitride.

Example 9 provides the sensor assembly of any of examples 6 to 8, wherein the FET is a graphene-FET such that the channel is a graphene channel, and the first layer is a hexagonal boron nitride layer formed on the channel of the FET.

Example 10 provides the sensor assembly of any preceding example, further comprising a second sensor responsive to the detection of an indicator of the presence of at least one amplification product to provide a second signal; and wherein the first sensor is a pH sensor adapted to detect a change in pH indicative of the presence of at least one amplification product and the second sensor is a sensor adapted to detect the presence of at least one amplification product directly.

Example 11 provides the sensor assembly of any preceding example, further comprising an optical sensor adapted to detect a change in optical properties corresponding to the presence of at least one amplification product.

Example 12 provides the sensor assembly of any preceding example, further comprising an ion selective membrane provided on the sensing surface.

Example 13 provides a system for isothermal amplification of a sample, the system comprising an isothermal amplification assembly for carrying out an isothermal amplification process on the sample to produce at least one amplification product; and a sensor assembly for detecting the presence of the at least one amplification product, the sensor assembly comprising: an amplification product receiving region; and a first sensor responsive to the detection of indicator of the presence of at least one amplification product to provide a first signal, the first sensor comprising a sensing surface arranged to contact a sample in the amplification product receiving region, wherein the sensing surface comprises a first layer comprising a one-dimensional or two-dimensional material.

Example 14 provides the system of example 13, wherein the sensor assembly is a sensor assembly according to any of examples 1 to 12.

Example 15 provides the system of example 14, wherein the one-dimensional or two-dimensional material is selected from graphene, hexagonal boron-nitride, carbon nano-tubes, or a combination thereof.

Example 16 provides the system of any of examples 13 to 15, wherein the one-dimensional or two-dimensional material is single-walled carbon nanotubes.

Example 17 provides the system of any of examples 13 to 16, wherein the first sensor is (i) a pH sensor adapted to detect a change in pH indicative of the presence of at least one amplification product; or (ii) a sensor adapted to detect the presence of at least one amplification product directly.

Example 18 provides the system of any of examples 13 to 17, further comprising a second sensor responsive to the detection of an indicator of the presence of at least one amplification product to provide a second signal; and wherein the first sensor is a pH sensor adapted to detect a change in pH indicative of the presence of at least one amplification product and the second sensor is a sensor adapted to detect the presence of at least one amplification product directly.

Example 19 provides the system of any of examples 13 to 18, wherein the isothermal amplification assembly is a loop assisted isothermal amplification (LAMP) assembly.

Example 20 provides a method for determining the presence of an analyte in a sample, the method comprising subjecting the sample to an isothermal amplification process, the isothermal amplification process producing at least one amplification product in the event that the analyte is present; providing the at least one amplification product to an amplification product receiving region; and detecting the presence of the at least one amplification product using a sensor assembly, the sensor assembly comprising a first sensor responsive to the detection of indicator of the presence of at least one amplification product to provide a first signal, the first sensor comprising a sensing surface arranged to contact a sample in the amplification product receiving region, wherein the sensing surface comprises a first layer comprising a one-dimensional or two-dimensional material.

Example 21 provides the method of example 20, further comprising: determining the presence of the at least one amplification product using a second sensor.

Example 22 provides the method of example 20 or example 21, wherein the sensor assembly is the sensor assembly according to any of examples 1 to 12.

Example 23 provides a pH sensor, comprising a sensing surface arranged to contact a sample, the sensing surface comprising a first layer comprising a one-dimensional or two-dimensional material, wherein pH sensor is adapted to provide a signal indicative of the pH of a sample.

Example 24 provides the pH sensor of example 23, wherein the one-dimensional or two-dimensional material is selected from graphene, hexagonal boron-nitride, carbon nano-tubes, or a combination thereof.

Example 25 provides the pH sensor of one of example 23 or example 24, wherein the one-dimensional or two-dimensional material is single-walled carbon nano-tubes.

Example 26 provides the pH sensor of any of examples 23 to 25, wherein the first layer is formed of the one-dimensional or two-dimensional material.

Example 27 provides the pH sensor of any of examples 23 to 26, wherein the first sensor an electrode-based sensor comprising at least one electrode adapted to provide the first signal.

Example 28 provides the pH sensor of any of examples 23 to 27, wherein the first sensor comprises a field effect transistor adapted to provide the first signal, and wherein the first layer is provided on a surface of the field effect transistor.

1. A sensor assembly for detecting the presence of at least one amplification product of an isothermal amplification process, the sensor assembly comprising:
an amplification product receiving region; and
a first sensor responsive to the detection of an indicator of the presence of at least one amplification product to provide a first signal, the first sensor comprising a sensing surface arranged to contact a sample in the amplification product receiving region,
wherein the sensing surface comprises a first layer comprising a one-dimensional or two-dimensional material; and
wherein the first sensor is either (i) a pH sensor adapted to detect a change in pH indicative of the presence of at least one amplification product; or (ii) a sensor adapted to detect the presence of at least one amplification product directly.

2. The sensor assembly of claim 1, wherein the one-dimensional or two-dimensional material is selected from graphene, hexagonal boron-nitride, carbon nano-tubes, or a combination thereof.

3. The sensor assembly of claim 2, wherein the one-dimensional or two-dimensional material is single-walled carbon nanotubes.

4. The sensor assembly of claim 1, wherein the first layer is formed of the one-dimensional or two-dimensional material.

5. The sensor assembly of any one of claims 1 to 4, wherein the first sensor is an electrode-based sensor comprising at least one electrode adapted to provide the first signal.

6. The sensor assembly of any one of claims 1 to 4, wherein the first sensor comprises a field effect transistor (FET) adapted to provide the first signal, and wherein the first layer is provided on a surface of the FET.

7. The sensor assembly of claim 6, wherein the first sensor further comprises a second layer beneath a channel of the field effect transistor, the second layer comprising or being formed of a one dimensional or two-dimensional material.

8. The sensor assembly of claim 6, wherein the first layer comprises or is formed of hexagonal-boron nitride.

9. The sensor assembly of claim 6, wherein the FET is a graphene-FET such that the channel is a graphene channel, and the first layer is a hexagonal boron nitride layer formed on the channel of the FET.

10. The sensor assembly of any one of claims 1 to 4, further comprising a second sensor responsive to the detection of an indicator of the presence of at least one amplification product to provide a second signal; and wherein the first sensor is a pH sensor adapted to detect a change in pH indicative of the presence of at least one amplification product and the second sensor is a sensor adapted to detect the presence of at least one amplification product directly.

11. The sensor assembly of any one of claims 1 to 4, further comprising an optical sensor adapted to detect a change in optical properties corresponding to the presence of at least one amplification product.

12. The sensor assembly of any one of claims 1 to 4, further comprising an ion selective membrane provided on the sensing surface.

13. A system for isothermal amplification of a sample, the system comprising:
an isothermal amplification assembly for carrying out an isothermal amplification process on the sample to produce at least one amplification product; and
a sensor assembly for detecting the presence of the at least one amplification product, the sensor assembly comprising:
an amplification product receiving region; and
a first sensor responsive to the detection of indicator of the presence of at least one amplification product to provide a first signal, the first sensor comprising a sensing surface arranged to contact a sample in the amplification product receiving region,
wherein the sensing surface comprises a first layer comprising a one-dimensional or two-dimensional material.

14. The system of claim 13, wherein the sensor assembly is a sensor assembly according to any of claims 1 to 12.

15. The system of claim 14, wherein the one-dimensional or two-dimensional material is selected from graphene, hexagonal boron-nitride, carbon nano-tubes, or a combination thereof.

16. The system of any one of claims 13 to 15, wherein the one-dimensional or two-dimensional material is single-walled carbon nanotubes.

17. The system of any one of claims 13 to 15, wherein the first sensor is (i) a pH sensor adapted to detect a change in pH indicative of the presence of at least one amplification product; or (ii) a sensor adapted to detect the presence of at least one amplification product directly.

18. The system of any one of claims 13 to 15, further comprising a second sensor responsive to the detection of an indicator of the presence of at least one amplification product to provide a second signal; and wherein the first sensor is a pH sensor adapted to detect a change in pH indicative of the presence of at least one amplification product and the second sensor is a sensor adapted to detect the presence of at least one amplification product directly.

19. The system of any one of claims 13 to 15, wherein the isothermal amplification assembly is a loop assisted isothermal amplification (LAMP) assembly.

20. A method for determining the presence of an analyte in a sample, the method comprising:
subjecting the sample to an isothermal amplification process, the isothermal amplification process producing at least one amplification product in the event that the analyte is present;
providing the at least one amplification product to an amplification product receiving region; and
detecting the presence of the at least one amplification product using a sensor assembly, the sensor assembly comprising a first sensor responsive to the detection of indicator of the presence of at least one amplification product to provide a first signal, the first sensor comprising a sensing surface arranged to contact a sample in the amplification product receiving region, wherein the sensing surface comprises a first layer comprising a one-dimensional or two-dimensional material.

21. The method of claim 20, further comprising:
determining the presence of the at least one amplification product using a second sensor.

22. The method of claim 20 or claim 21, wherein the sensor assembly is the sensor assembly according to any of claims 1 to 12.

23. A pH sensor, comprising:
a sensing surface arranged to contact a sample, the sensing surface comprising a first layer comprising a one-dimensional or two-dimensional material,
wherein pH sensor is adapted to provide a signal indicative of the pH of a sample.

24. The pH sensor of claim 23, wherein the one-dimensional or two-dimensional material is selected from graphene, hexagonal boron-nitride, carbon nano-tubes, or a combination thereof.

25. The pH sensor of claim 23, wherein the one-dimensional or two-dimensional material is single-walled carbon nano-tubes.

26. The pH sensor of any one of claims 23 to 25, wherein the first layer is formed of the one-dimensional or two-dimensional material.

27. The pH sensor of any one of claims 23 to 25, wherein the first sensor an electrode-based sensor comprising at least one electrode adapted to provide the first signal.

28. The pH sensor of any one of claims 23 to 25, wherein the first sensor comprises a field effect transistor adapted to provide the first signal, and wherein the first layer is provided on a surface of the field effect transistor.
